(19) United States
(12) Patent Application Publication
(10) Pub. No.: US 2020/0146721 A1
(43) Pub. Date: May 14, 2020

(54) Threaded Joint for Supporting and Stabilizing a Rear Fracture of the Pelvic Ring or for Stabilizing and Stiffening the Sacroiliac Joint

(71) Applicant: Nazar Sadiq, Wilkau-Hasslau (DE)

(72) Inventor: Nazar Sadiq, Wilkau-Hasslau (DE)

(21) Appl. No.: 16/614,354

(22) PCT Filed: May 29, 2018
(86) PCT No.: PCT/DE2018/000167

§ 371 Date: Nov. 16, 2019

(30) Foreign Application Priority Data
May 31, 2017 (DE) 10 2017 005 222.0
May 31, 2017 (DE) 20 2017 002 906.5

(51) Int. Cl. A61B 17/68; A61B 17/70; A61B 17/86

(52) U.S. Cl. CPC A61B 17/683; A61B 17/7055; A61B 17/8695; A61B 17/8605; A61B 17/8665

(57) ABSTRACT

Threaded joints for supporting and stabilizing a rear fracture of the pelvic ring or for stabilizing and stiffening the sacroiliac joint, either with a threaded bar in connection with threaded nuts or a screw in connection with the sacrum. The threaded joints are characterized by secure support and stabilization of a rear fracture of the pelvic ring or by secure stabilization and stiffening of the sacroiliac joint. The threaded joint as an implant is either a threaded bar in connection with threaded nuts for supporting the rear fracture of the pelvic ring or a screw in connection with the sacrum. There is a washer located between the ilium and the threaded nut or the screw head, the side of the washer that faces the ilium being mutually oblique in at least two planes and the oblique surfaces being arranged at an angle to each another.

### CROSS REFERENCE TO RELATED APPLICATIONS

This application is the U.S. national stage of International Application No. PCT/DE2018/000167, filed on 2018 May 29. The international application claims the priority of DE 102017005222.0 filed on 2017 May 31 and the priority of DE 202017002906.5 filed on 2017 May 31; all applications are incorporated by reference herein in their entirety.

### BACKGROUND

The invention relates to threaded joints for supporting and stabilising a posterior pelvic ring fracture or for stabilising and stiffening the sacroiliac joint, either using a threaded rod in conjunction with threaded nuts, or a screw in conjunction with the sacrum.

DE 10 2013 005 391 A1 discloses a system for supporting a degenerative sacroiliac joint, which system has a tensioning element which is held between the left and right pelvic bones by means of a holding device. For this purpose, the holding device has a support which extends through a bore in the relevant pelvic bone, and a relevant end piece.

U.S. Pat. No. 6,635,059 B2 contains a screw system for fixing the sacrum and/or the sacroiliac joint, and/or for use in other skeletal regions.

In both solutions, simple washers are used as planar surface structures. When using washers of this kind, the compression force of the screw is concentrated on a small bone surface of the ilium owing to the shape of the bone surface. As a result, a fracture of the outer cortical bone of the ilium may occur. Furthermore, a loss of stability is possible, in particular when the os sacrum has a poor bone quality, for example due to osteoporosis and bone tumours. When using a rigid rod which has simple washers, an additional fracture in the region of the greater sciatic notch often occurs as a post-operative complication. This leads to a loss of stability in the region of the original os sacrum fracture, persistent pain symptoms, and possibly to pseudarthrosis and to the possible failure of the surgical treatment.

US 2015/0320450 A1 discloses a sacral fixation system, in which in particular a stabilisation of the pelvic ring or of the sacroiliac joint is intended to be achieved. The system comprises a threaded rod in conjunction with threaded nuts, with a washer being located between the threaded nut and the bone. The face of the washer that faces the ilium is mutually inclined in at least two planes. Furthermore, the washer has an opening for the passage of a region of the threaded rod. When using washers of this kind, the compression force of the screw is likewise concentrated on a small bone surface of the ilium.

### SUMMARY

The invention relates to threaded joints for supporting and stabilising a rear fracture of the pelvic ring or for stabilising and stiffening the sacroiliac joint, either with a threaded bar in connection with threaded nuts or a screw in connection with the sacrum. The threaded joints are characterised by secure support and stabilisation of a rear fracture of the pelvic ring or by secure stabilisation and stiffening of the sacroiliac joint. The threaded joint as an implant is, for this purpose, either a threaded bar in connection with threaded nuts for supporting the rear fracture of the pelvic ring or a screw in connection with the sacrum. For this purpose, there is a washer located between the ilium and the threaded nut or the screw head, the side of the washer that faces the ilium being mutually oblique in at least two planes and the oblique surfaces being arranged at an angle to each another. The washer has an opening for the passage of a region of the threaded bar or of the screw, the opening being expanded towards the ilium.

### DETAILED DESCRIPTION

The problem addressed by the invention specified in claim 1 is that of providing a threaded joint as an implant that can be easily and securely placed, for supporting a posterior pelvic ring fracture or for stabilising the sacroiliac joint.

This problem is solved by the features listed in claim 1.

The threaded joints are characterised by secure support and stabilisation of a posterior pelvic ring fracture, or by secure stabilisation and stiffening of the sacroiliac joint. The threaded joint as an implant is either a threaded rod in conjunction with threaded nuts for supporting the posterior pelvic ring fracture, or a screw in conjunction with the sacrum.

For this purpose, a washer is located between the ilium and the threaded nut or the screw head, the face of the washer that faces the ilium being mutually inclined in at least two planes and the inclines being arranged at an angle to one another. The washer has an opening for the passage of a region of the threaded rod or of the screw, the opening being widened in the direction of the ilium.

The threaded joint is a surgical implant for stabilising the posterior pelvic ring in the case of a fracture of the sacrum and in the case of an existing pathology in the region of the sacroiliac joint. The implant can remain permanently or at least for a defined period of time. Sufficient or maximum stability is achieved in the region of the fracture using the threaded joint, in order to prevent the formation of a false joint (pseudarthrosis) and to achieve a rapid, load-stable treatment of the fracture.

The secure support of the posterior pelvic ring fracture or the secure stabilisation of the sacroiliac joint is ensured in particular by the anatomically shaped washer which has the special opening. The washer is formed with reference to the geometry and angles of the ilium in the region of the ala ossis ilii and the sacrum. For this purpose, the face of the washer that faces the ilium is mutually inclined in at least two planes, the inclines further being arranged at an angle to one another. The surface inclination and the angle of the face are adapted to the anatomy of the ilium. For this purpose, the planes can be designed to be tabular and/or spherical, regular or irregular. The inclines can also be planar and/or convex. The compression forces can therefore be evenly distributed over the outer cortical bone of the ilium by means of the threaded rod and/or the screw during the fracture stabilisation of the sacral fracture or during the stiffening of the sacroiliac joint in the sense of an arthrodesis.

The opening of the washer which is widened in the direction of the ilium allows an exact placement of the threaded rod or the screw.

The threaded rod is for this purpose a rod, of which at least the end regions have a thread for engaging the nuts as threaded nuts. Said rod can advantageously be a titanium rod. Said rod is simply placed in a transsacral manner in conjunction with the anatomically shaped washers. For this purpose, the threaded rod is located in a formed bore. Due to the material properties and geometry of the threaded rod in conjunction with the washers, a good but not rigid compression is achieved in the region of the outer cortical bone of the ilium. Complications in the form of a fracture in the region of the greater sciatic notch, and a fracture of the outer cortical bone of the ilium are largely preventable. This is further achieved by the anatomically shaped faces of the washer, such that a distribution of the compression forces over a large bone surface of the ilium is achieved. This prevents a fracture in the region of the outer cortical bone of the ilium.

Thus, the threaded joint is advantageously suitable for stabilising the traumatic fracture of the os sacrum of all types according to Denis I, II and III for mono- and bilateral fractures, in particular for os sacrum fractures which have reduced bone quality and pathological fractures, for example in the case of osteoporosis and osteopenia, and pathological fractures caused by primary tumours or bone metastasis. The pseudarthrosis can also be treated after failed conservative or surgical treatment of the os sacrum fractures, by achieving the sufficient stability of the bones in the region of the fracture. In addition, it is possible to treat arthrodesis in the case of primary and secondary arthrosis of the sacroiliac joint and in the case of posttraumatic degeneration and osteoligamentous dysfunction of the sacroiliac joint.

Advantageous embodiments of the invention are specified in claims 2 to 12.

The faces and the openings of the washer that face the ilium mirror one another for placement on the left ilium and on the right ilium, according to the development of claim 2.

According to the development of claim 3, the incline is a straight line or an arch in cross section.

According to the development of claim 4, the inner wall of the opening is a cone.

According to the development of claim 5, the face of the washer that faces the ilium has at least one elongate recess for supporting the attachment of the washer to the periosteum. The elongate recess leads to improved adhesion of the washer to the periosteum.

According to the development of claim 6, the screw has a conical screw head, which allows the alignment and placement of the screw through the opening in the washer into the sacrum in the case of normal anatomy and in the case of dysmorphism of the sacrum. This is achieved by means of the conical screw head, which allows different inclinations of the screw. The screw can therefore be placed in the os sacrum in the case of normal anatomy and in the case of dysmorphism of the os sacrum.

According to the development of claim 7, the screw has the conical screw head, a cylindrical shank and a thread. Damage to the inner wall of the opening in the washer is prevented by the cylindrical shank.

According to the development of claim 8, the screw has a continuous channel having at least one outlet opening in the region of thread. The channel in the screw can be used for placing the screw in the os sacrum by means of a guide wire and/or for applying bone cement when augmenting the screw.

According to the development of claim 9, the face of the washer that faces away from the ilium has at least two of the holes which are used for positioning on the hip bone in order to releasably engage projections of a positioning sleeve. This is used for optimum placement of the anatomically shaped washer on the outer cortical bone of the ilium, in particular using a minimally invasive technique.

According to the development of claim 10, the projections are axial extensions of the positioning sleeve.

According to the development of claim 11, the inclines of the washer are adapted to the ilium in the region of the ala ossis

According to the development of claim 12, the washer and an alignment arm of the positioning sleeve connected to the washer are arranged such that the alignment arm points towards the ventral edge of the joint socket of the hip joint. An accurate placement and alignment of the washers on the right and left sides of the os ilium is therefore provided. Intraoperative X-ray control of the position of the washer, which would otherwise be required, is therefore minimised or even avoided. The optimal placement of the anatomically shaped washer on the os ilium is provided when the alignment arm is oriented towards the ventral edge of the joint socket of the hip joint. In this case, the surface of the face of the washer that faces the ilium matches the anatomy of the os ilium in the region of the ala ossis

One embodiment of the invention is shown schematically in each of the drawings and is described in greater detail below.

### BRIEF DESCRIPTION OF THE DRAWINGS

In the drawings:

FIG. 1 shows a threaded joint for supporting a posterior pelvic ring fracture,

FIG. 2 shows a side view of the pelvis with a washer on the ilium,

FIG. 3 shows a washer of a threaded joint for supporting a posterior pelvic ring fracture or for stabilising the sacroiliac joint,

FIG. 4 shows a plan view of a washer,

FIG. 5 shows washers of a threaded joint for supporting a posterior pelvic ring fracture,

FIG. 6 shows a sectional view of a washer having two holes which are used for positioning on the hip bone in order to releasably engage projections of a positioning sleeve

FIG. 7 shows a washer having a positioning sleeve,

FIG. 8 shows a screw for stabilising the sacral fracture and/or stiffening the sacroiliac joint, and

FIG. 9 shows a sectional view of a screw for stabilising the sacral fracture and/or stiffening the sacroiliac joint.

### DETAILED DESCRIPTION OF THE PREFERRED EMBODIMENTS

A threaded joint for supporting a posterior pelvic ring fracture substantially consists of a threaded rod 1 in conjunction with threaded nuts and anatomically shaped washers 2.

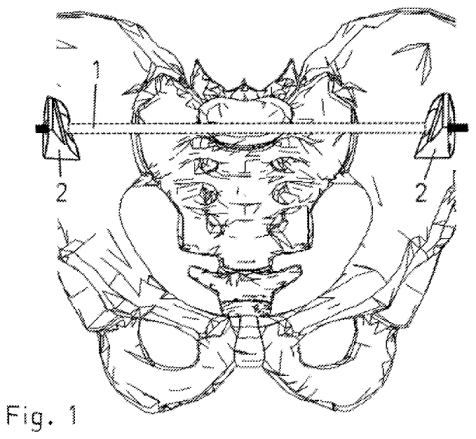

FIG. 1 schematically shows a threaded joint for supporting a posterior pelvic ring fracture.

The washers 2 are located between the ilium of the pelvis and the known threaded nuts. The threaded nuts are not shown in FIG. 1.

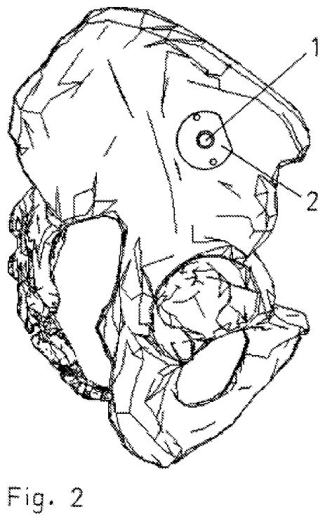

FIG. 2 schematically shows a side view of the pelvis having a washer 2 on the ilium.

One washer 2 rests on the ilium in each case.

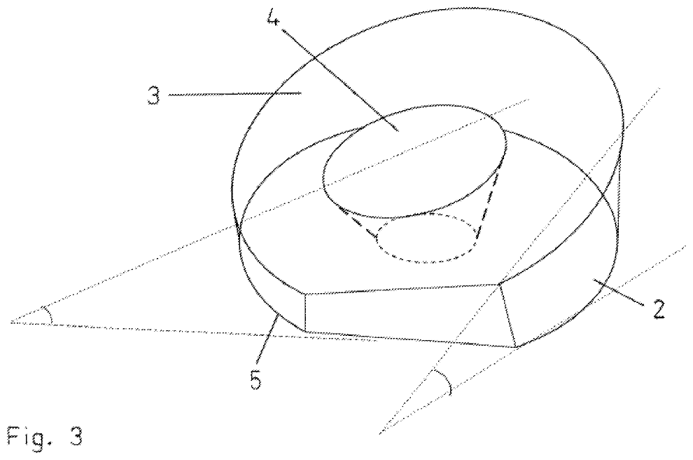

FIG. 3 schematically shows a washer 2 of a threaded joint for supporting a posterior pelvic ring fracture or for stabilising the sacroiliac joint.

The face 3 of the washer 2 that faces the ilium is mutually inclined in at least two planes, the inclines being arranged at an angle to one another. The incline is a straight line and/or an arch in cross section. The washer 2 also has an opening 4 for the passage of a region of the threaded rod 1 or of a screw 11. The opening 4 is widened in the direction of the ilium, and in particular is conical. The face 5 of the washer 2 that is opposite the ilium, and the threaded rod 1 which passes through the opening 4 form a right angle or an approximate right angle.

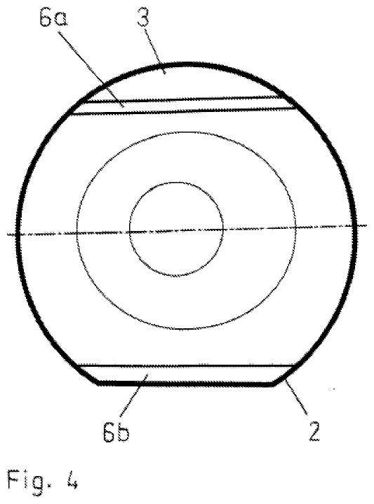

FIG. 4 schematically shows a plan view of a washer 2.

The face 3 of the washer 2 that faces the ilium has two elongate recesses for supporting the attachment of the washer 2 to the periosteum. The recesses are grooves 6, it being possible for one groove 6a to have a V-shape in cross section, and the other groove 6b to have an L-shape in cross section.

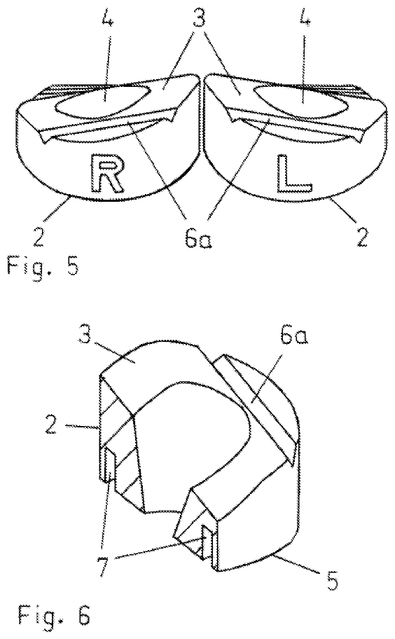

FIG. 5 schematically shows washers 2 of a threaded joint for supporting a posterior pelvic ring fracture.

The faces 3 and openings 4 of the washers 2 that face the ilium mirror one another for placement on the left ilium and on the right ilium. For this purpose said washers can be marked with an R for right and an L for left.

FIG. 6 schematically shows a sectional view of a washer 2 having two of the holes 7 which are used for positioning on the ilium in order to releasably engage projections of a positioning sleeve.

The face 5 of the washer 2 that faces away from the hip bone has at least two of the holes 7 which are used for positioning on the hip bone in order to releasably engage projections 9 of a positioning sleeve 8.

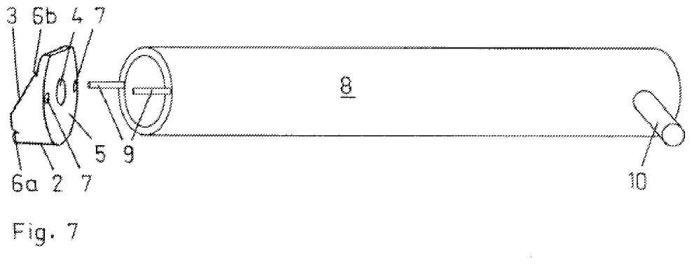

FIG. 7 schematically shows a washer 2 having the positioning sleeve 8.

The projections 9 are axial extensions of the positioning sleeve 8. The inclines of the washer 2 are adapted to the ilium in the region of the ala ossis ilii. The washer 2 and an alignment arm 10 of the positioning sleeve 8 connected to the washer 2 are arranged such that the alignment arm 10 points towards the ventral edge of the joint socket of the hip joint.

In one embodiment, a threaded joint for stabilising the sacroiliac joint can consist of a washer 2 and a screw 11 in conjunction with the sacrum.

For this purpose, the opening 4 of the washer 2 is used for the passage of the screw 11.

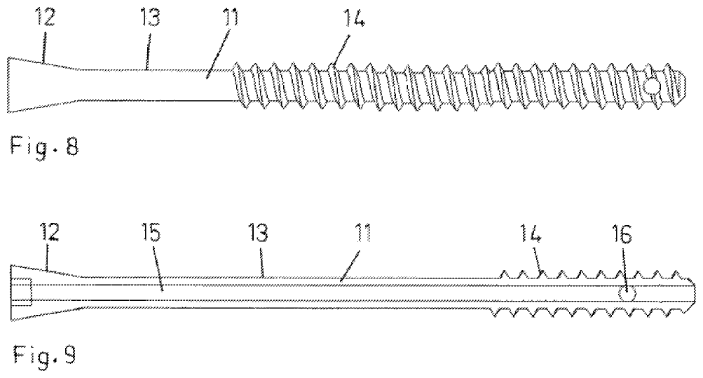

FIG. 8 schematically shows a screw 11 for stabilising the sacral fracture and/or stiffening the sacroiliac joint.

The screw 11 has a conical screw head 12, which allows the alignment and placement of the screw 11 through the opening 4 in the washer 2 into the sacrum, in the case of normal anatomy and in the case of dysmorphism of the sacrum. Furthermore, the screw 11 has a cylindrical shank 13 and a thread 14.

FIG. 9 schematically shows a sectional view of a screw 11 for stabilising the sacral fracture and/or stiffening the sacroiliac joint.

The screw 11 has a continuous channel 15 having at least one outlet opening 16 in the region of the thread 14.

1. Threaded joint for supporting and stabilising a posterior pelvic ring fracture, or for stabilising and stiffening the sacroiliac joint, either using a threaded rod (1) in conjunction with threaded nuts, or a screw (11) in conjunction with the sacrum, characterised in that a washer (2) is located between the ilium and the threaded nut or the screw head, in that the face (3) of the washer (2) that faces the ilium is
mutually inclined in at least two planes, the inclines being arranged at an angle to one another, and in that the washer (2) has an opening (4) for the passage of a region of the threaded rod (1) or of the screw (11), the opening (4) being widened in the direction of the ilium.

2. Threaded joint according to claim 1, characterised in that the faces (3) and the openings (4) of the washer (2) that face the ilium mirror one another for placement on the left ilium and on the right ilium.

3. Threaded joint according to claim 1, characterised in that the incline is a straight line or an arch in cross section.

4. Threaded joint according to claim 1, characterised in that the inner wall of the opening (4) is a cone.

5. Threaded joint according to claim 1, characterised in that the face (3) of the washer (2) that faces the ilium has at least one elongate recess for supporting the attachment of the washer (2) to the periosteum.

6. Threaded joint according to claim 1, characterised in that the screw (11) has a conical screw head (12).

7. Threaded joint according to claim 6, characterised in that the screw (11) has the conical screw head (12), a cylindrical shank (13) and a thread (14).

8. Threaded joint according to claim 6, characterised in that the screw (11) has a continuous channel (15) having at least one outlet opening (16) in the region of the thread (14).

9. Threaded joint according to claim 1, characterised in that the face (5) of the washer (2) that faces away from the ilium has at least two of the holes (7) which are used for positioning on the ilium in order to releasably engage projections (9) of a positioning sleeve (8).

10. Threaded joint according to claim 9, characterised in that the projections (9) are axial extensions of the positioning sleeve (8).

11. Threaded joint according to claim 1, characterised in that the inclines of the washer (2) are adapted to the ilium in the region of the ala ossis ilii.

12. Threaded joint according to claim 1, characterised in that the washer (2) and an alignment arm (10) of the positioning sleeve (8) which is connected to the washer (2) are arranged such that the alignment arm (10) points towards the ventral edge of the joint socket of the hip joint.
